(19) United States
(12) Patent Application Publication
(10) Pub. No.: US 2018/0296458 A1
(43) Pub. Date: Oct. 18, 2018

(54) COSMETIC COMPOSITION COMPRISING AT LEAST ONE LAMBDA-CARRAGEENAN POLYSACCHARIDE IN COMBINATION WITH AT LEAST ONE SPECIFIC POLYOL, AND PROCESS FOR COSMETIC TREATMENT OF KERATIN FIBERS WITH THE COMPOSITION, AND USE OF THE COMPOSITION FOR HAIR CARE

(71) Applicant: L'OREAL, Paris (FR)

(72) Inventors: Ludivine LAURENT, Courbevoie (FR); Cécile BEBOT, Clichy (FR)

(21) Appl. No.: 16/015,806

(22) Filed: Jun. 22, 2018

Related U.S. Application Data
(63) Continuation of application No. 11/882,779, filed on Aug. 6, 2007, now abandoned.

(30) Foreign Application Priority Data
Aug. 4, 2006 (FR) 0607157

(51) Int. Cl. A61K 8/73; A61Q 5/06; A61K 8/34

(52) U.S. Cl. CPC A61K 8/73; A61Q 5/06; A61K 8/345

(57) ABSTRACT

The present disclosure relates to a cosmetic composition for the treatment of keratin fibers, for instance human keratin fibers such as the hair, comprising at least one lambda-carrageenan polysaccharide, at least one polyol containing in its structure at least 3 hydroxyl groups, other than carrageenans, and at least one specific additive, to a cosmetic treatment process using the composition, and to the use of this composition for fixing and caring for human keratin fibers.

This application claims benefit of priority under 35 U.S.C. § 119 to French Patent Application No. FR 06/007157, filed Aug. 4, 2006, the contents of which are also incorporated herein by reference.

The present disclosure relates to a cosmetic composition for the treatment of keratin fibers, for instance human keratin fibers such as the hair, comprising at least one lambda-carrageenan polysaccharide, at least one specific polyol and at least one specific additive, to a process for cosmetic treatment using the composition, and to the use of this composition for hair care.

In the hairstyling field, for instance among hair products for use in shaping and/or holding the hairstyle, hair compositions generally comprise a solution, most commonly alcoholic or aqueous, of at least one fixing polymer as a mixture with various cosmetic adjuvants.

These compositions can be in the form of hair gels or mousses which are generally applied to wetted hair before blow-drying or drying is carried out.

Hair gels may comprise at least one thickening polymer or gelling agent in combination with at least one fixing polymer which most commonly have the function of forming a film at the surface of the keratin fibers to be fixed.

Recently, carrageenans have been used as fixing polymers in hairstyling gels or in aerosols. In fact, it is known practice, from document EP 1 199 064, to use a carrageenan or a mixture thereof and a specific additive in order to obtain a solid and stable gel for hair treatment.

Carrageenans are polysaccharides which constitute the cell walls of various red algae (Rhodophyceae) belonging to the Gigartinaceae, Hypneaceae, Furcellariaceae and Polyideaceae families. They comprise long anionic polyelectrolyte galactanic chains. Their molecular mass can be greater than 10⁶. These linear polymers, formed from disaccharide units, are composed of two D-galactopyranose units alternately linked by α- and β-linkages. They are highly sulphated (20-50%) polysaccharides and the α-D-galactopyranosyl residues can be in 3′,6′-anhydro form.

Initially, carrageenans were divided up into two families according to their solubility in potassium chloride (KCl). The fractions soluble in KCl were denoted by the prefix “kappa”, while the term “lambda” was reserved for those which are insoluble. Later on, the classifications were based on the number and the position of sulphate groups and also the presence of a 3′,6′-anhydro bridge on the β-D-galactopyranosyl residues. This resulted in four major families: κ, λ, β, ω.

The various types of carrageenans do not exist in the pure state, but in the form of hybrids. Thus, in the natural state, κ- and ι-carrageenans are in the form of a kappa-iota hybrid, but one of the two structures may predominate over the other. The κ-ι hybrid state of a structure can be elucidated using specific enzymes which make it possible to enrich or decrease the content of one of the two forms. Carrageenans can coexist with their precursors. Carrageenans belonging to various families can coexist in a hybrid structure; for example: carrageenan of Euchema gelatinae: a hybrid of β-carrageenan, the major component, and of κ, γ-carrageenans.

The use, in hair care, of a λ-carrageenan type polysaccharide makes it possible to obtain hairstyling gels with good fixing properties. However, the film formed at the surface of the hair is quite friable and does not allow the shaping to be held over a long period of time.

There exists, therefore, a real need to find cosmetic compositions, for instance for hairstyling, which make it possible to improve the properties of hold of the hairstyle over time.

Surprisingly and unexpectedly, the present inventors have discovered that, by combining at least one λ-carrageenan with specific polyols and specific additives, it is possible to obtain compositions whose hold over time may be improved, while at the same time obtaining a very limited friability of the fixing film, solving one or more of the problems mentioned above. Further, it has been observed that, in this composition, the lambda-carrageenans make it possible to obtain gels which have a better texture, which are less brittle and less hard and which are easier to apply.

One aspect of the present disclosure is therefore a cosmetic composition for the treatment of keratin fibers, for instance human keratin fibers such as the hair, comprising, in a cosmetically acceptable medium:
at least one lambda-carageenan polysaccharide;
at least one specific polyol other than carrageenans; and
at least one additive chosen from a silicone, a fatty substance and a fixing polymer other than carrageenans.

Another aspect of the present disclosure is a cosmetic treatment process using the cosmetic composition as disclosed herein.

A further aspect of the present disclosure is the use of the cosmetic composition as disclosed herein for fixing the hair or for hair care.

Other subjects, characteristics, aspects and benefits of the present disclosure will emerge more clearly on reading the description and the examples which follow.

As disclosed herein, the word “hairstyling” is understood to mean the fact that the shape of the hairstyle is fixed and/or held.

The composition according to the present disclosure comprises at least one lambda-carrageenan polysaccharide.

According to at least one embodiment, the lambda-carrageenan polysaccharide used according to the present disclosure is not chemically modified.

For instance, according to at least one embodiment, the molecular weight (MW) of the polysaccharide ranges from 100,000 to 1,000,000. Even further, for example, the molecular weight ranges from 250,000 to 800,000.

By way of lambda-carrageenan polysaccharide that may be used in the context of the present disclosure, non-limiting mention may be made of Satiagum UTC 10 from the company Degussa and Welgeenan ED 1039 from the company Eurogum.

The lambda-carrageenan polysaccharide can be present in the composition in an amount ranging from 0.1% to 30%, for instance from 0.2% to 20%, and even further, for example, from 0.5% to 15% by weight, relative to the total weight of the cosmetic composition.

The polyols that can be used in the composition according to the present disclosure contain, in their structure, at least three hydroxyl (OH) groups.

For instance, the polyols according to the present disclosure can be chosen from simple polyols, monosaccharides, disaccharides, oligosaccharides and polysaccharides other than carrageenans.

As used herein, the term “simple polyols” is understood to mean compounds comprising a linear or branched alkyl or alkenyl chain containing from 3 to 100 carbon atoms, comprising at least three hydroxyl (OH) groups. According to at least one embodiment, this alkyl or alkenyl chain is C₃-C₂₀.

By way of example of simple polyols, non-limiting mention may be made of glycerol or 1,2,3-propanetriol, 1,2,3-butanetriol, sorbitol, 3-methyl-1,3,5-pentanetriol and 1,2,4-butanetriol.

By way of example of polysaccharides other than carrageenans, non-limiting mention may be made of:
maize starch, rice starch, cassava starch, tapioca starch, oat starch, barley starch, potato starch, wheat starch, sorghum starch and pea starch, for example in the form of an insoluble white powder, in which the size of the elemental particles ranges from 3 to 100 microns, starches modified by at least one of the following reactions: pregelatinization, oxidation, crosslinking, for example with phosphated monostarch phosphate, distarch phosphate or even tristarch phosphate compounds or mixtures thereof, esterification, thermal treatments. The tapioca dextrin sold under the name Crystal Tex 626 by the company National Starch or crosslinked potato carboxymethyl starch sold under the name Primojel by the company DMV will, for example, be used.

Use may be made, for instance, of distarch phosphates or compounds rich in distarch phosphate, such as the product proposed under the references Prejel VA-70-T AGGL (gelatinized, hydroxypropylated cassava distarch phosphate) or Prejel TK1 (gelatinized cassava distarch phosphate) or Prejel 200 (gelatinized acetylated cassava distarch phosphate) by the company Avebe or Structure Zea from National Starch (gelatinized maize distarch phosphate).

Amphoteric starches can also be used; these amphoteric starches comprise at least one anionic group and at least one cationic group. The at least one anionic and cationic groups can be linked to the same reactive site of the starch molecule or to different reactive sites; according to at least one embodiment, they are linked to the same reactive site; and include, for instance, chemically modified or nonmodified, nonionic guar gums. The nonmodified, nonionic guar gums are, for example, the products sold under the name Vidogum GH 175 by the company Unipectine and under the name Jaguar C by the company Meyhall.

The modified nonionic guar gums that can be used according to the present disclosure are, for instance, modified with C₁-C₆ hydroxyalkyl groups. By way of example, non-limiting mention may be made of hydroxymethyl, hydroxyethyl, hydroxypropyl and hydroxybutyl groups.

These guar gums are well known from the prior art and can, for example, be prepared by reacting corresponding alkene oxides, for instance propylene oxides, with the guar gum so as to obtain a guar gum modified with hydroxypropyl groups.

Such nonionic guar gums optionally modified with hydroxyalkyl groups are, for example, sold under the trade names Jaguar HP8, Jaguar HP60, Jaguar HP120, Jaguar DC 293 and Jaguar HP 105 by the company Meyhall, or under the name Galactasol 4H4FD2 by the company Aqualon.

The alkyl radicals of the nonionic polymers have from 1 to 6 carbon atoms unless otherwise mentioned;
celluloses and their chemically modified derivatives such as hydroxyalkylcelluloses, carboxymethylcelluloses, cationized celluloses such as the product sold under the trade name JR400 by the company Dow Chemical;
cationic polysaccharides, for example, those with a quaternary ammonium, such as those described in U.S. Pat. Nos. 3,589,578 and 4,031,307, for instance guar gums comprising trialkylammonium cationic groups. For example, such products are sold under the trade names Jaguar C13 S, Jaguar C 15 and Jaguar C 17 by the company Meyhall.

Non-limiting mention may also be made of gums of biopolysaccharides of microbial origin, such as scleroglucan gum or xanthan gum.

Also suitable are gums derived from plant exudates, such as gums arabic, ghatti gums, karaya and tragacanth gums; hydroxypropylcelluloses or carboxymethylcelluloses; pectins and alginates.

These polymers are well known to those skilled in the art and are described, for instance, in the book by Robert L. Davidson entitled “Handbook of Water Soluble Gums and Resins” published by McGraw Hill Book Company (1980).

The composition may similarly comprise polymers derived from associative celluloses, such as:
quaternized cationic celluloses modified with groups comprising at least one hydrophobic chain, such as alkyl, arylalkyl or alkylaryl groups containing at least 8 carbon atoms, or mixtures thereof, or
cationic cellulose derivatives, such as copolymers of cellulose or of cellulose derivatives grafted with a water-soluble monomer comprising a quaternary ammonium, and described, for example, in U.S. Pat. No. 4,131,576, such as hydroxyalkylcelluloses, for instance hydroxymethyl-, hydroxyethyl- or hydroxypropylcelluloses grafted for instance with a methacryloyloxyethyltrimethylammonium salt, methacrylamidopropyltrimethylammonium salt or dimethyldiallylammonium salt.

According to at least one embodiment, the alkyl radicals borne by the quaternized celluloses or hydroxyethylcelluloses comprise 8 to 30 carbon atoms, and the aryl radicals are chosen, for instance, from phenyl, benzyl, naphthyl and anthryl groups.

As examples of quaternized alkylhydroxyethylcelluloses comprising a C₈-C₃₀ hydrophobic chain, non-limiting mention may be made of the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by the company Amerchol, and the products Crodacel QM®, Crodacel QL® (C₁₂ alkyl) and Crodacel QS® (C₁₈ alkyl) sold by the company Croda;
nonionic cellulose derivatives such as hydroxyethylcelluloses modified with groups comprising at least one hydrophobic chain, for instance alkyl, arylalkyl or alkylaryl groups, or mixtures thereof, and in which the alkyl groups are, for example, C₈-C₂₂, such as the product Natrosol Plus Grade 330 CS® (C₁₆ alkyls) sold by the company Aqualon, or the product Bermocoll EHM 100® sold by the company Berol Nobel;
cellulose derivatives modified with polyalkylene glycol alkyl phenol ether groups, such as the product Amercell Polymer HM-1500® sold by the company Amerchol.

According to at least one embodiment, the polyol is not a polymer.

The polyol(s) can be present in the composition in an amount ranging from 0.1% to 30%, for instance from 0.2% to 20%, and even further, for example, from 0.5% to 15% by weight, relative to the total weight of the cosmetic composition.

The additives usable in the composition according to the present disclosure are chosen from a silicone, a fatty substance and a fixing polymer other than carrageenans.

The cosmetic compositions in accordance with the present disclosure can be in the form of a cream, a mousse, a paste or a gel. According to at least one embodiment, this gel has a viscosity of greater than 200 cps at 25° C. at a shear rate of 1 s⁻¹. For instance, this viscosity ranges from 500 to 500,000 cps at 25° C., such as from 100,000 cps at 25° C., and even further, for example, from 500 to 50,000 cps at 25° C., at a shear rate of 1 s⁻¹. It can be measured using a viscometer of cone/plate type.

The cosmetic composition according to the present disclosure can comprise at least one silicone.

As used herein, the term “silicone” is understood to refer to any organosilicone polymers or oligomers with a linear or cyclic, branched or crosslinked structure, of variable molecular weight, obtained by polymerization and/or by polycondensation of suitably functionalized silanes, and comprising a repetition of principle units in which the silicone atoms are linked to one another via oxygen atoms (—Si—O—Si-siloxane linkage), optionally substituted hydrocarbon-based radicals being directly linked, via a carbon atom, to said silicone atoms. For example, the most common hydrocarbon-based radicals are alkyl radicals, such as C₁-C₁₀, and further, for instance, methyl radicals, fluoroalkyl radicals in which the alkyl part is C₁-C₁₀, aryl radicals, and phenyl radicals.

According to at least one embodiment, the silicone is an oxyalkylenated silicone.

As disclosed herein, the term “oxyalkylenated silicone” is understood to mean any silicone comprising at least one oxyalkylenated group of type (—CₓH₂ₓO—)ₐ in which x can range from 2 to 6, and a is greater than or equal to 2.

The oxyalkylenated silicones that can be used in the cosmetic composition may be chosen from the following formulae (VI), (VII), (VIII) and (IX):

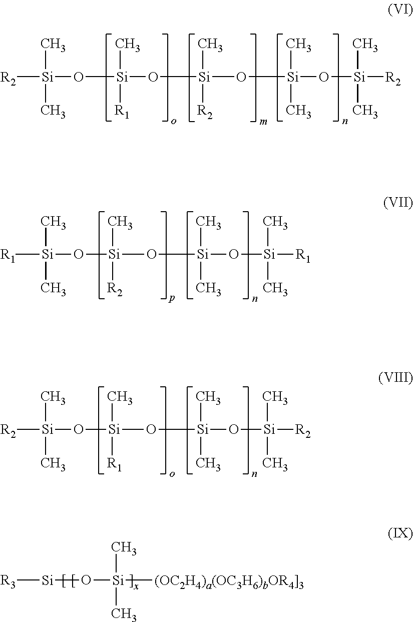

wherein:
R₁, which may be identical or different, is a linear or branched C₁-C₃₀ alkyl radical or phenyl radical;
R₂, which may be identical or different, is chosen from a —C_{c}H_{2c}—O—(C₂H₄O)ₐ(C₃H₆O)_{b}—R₅ radical and a —C_{c}H_{2c}—O—(C₄H₈O)ₐ—R₅ radical;
R₃ and R₄, which may be identical or different, are linear or branched C₁ to C₁₂ alkyl radicals, for instance methyl radicals;
R₅, which may be identical or different, is chosen from a hydrogen atom, a linear or branched C₁ to C₁₂ alkyl radical, a linear or branched C₁ to C₆ alkoxy radical, a linear or branched C₂ to C₃₀ acyl radical, a hydroxyl radical, —SO₃M, C₁-C₆ aminoalkoxy optionally substituted on the amine, C₂-C₆ aminoacyl optionally substituted on the amine; —NHCH₂CH₂COOM, —N(CH₂CH₂COOM)₂, aminoalkyl optionally substituted on the amine and on the alkyl chain, C₂-C₃₀ carboxyacyl, a group optionally substituted with one or two substituted aminoalkyl radicals, —CO(CH₂)_{d}COOM, —COCHR₇(CH₂)_{d}COOM, —NHCO(CH₂)_{d}OH, —NH₃Y, and a phosphate group;
M, which may be identical or different, is chosen from a hydrogen atom, Na, K, Li, NH₄ and an organic amine;
R₇ is a hydrogen atom or an SO₃M radical;
d ranges from 1 to 10;
m ranges from 0 to 20;
n ranges from 0 to 500;
o ranges from 0 to 20;
p ranges from 1 to 50;
a ranges from 0 to 50;
b ranges from 0 to 50;
a+b is greater than or equal to 2;
c ranges from 0 to 4;
x ranges from 1 to 100; and
Y is a monovalent inorganic or organic anion such as halide (chloride, bromide), sulphate or carboxylate (acetate, lactate, citrate);
with the proviso that, when the silicone is of formula (VII) with R₅ denoting hydrogen, then n is greater than 12.

Such silicones are, for example, sold by the company Goldschmidt under the trade names Abil WE 09, Abil EM 90, Abil B8852, Abil B8851, Abil B 8843 and Abil B8842, by the company Dow Corning under the names Fluid DC 190, DC 3225 C, Q2-5220, Q25354 and 02-5200, by the company Rhodia Chimie under the names Silbione Huile 70646 and Rhodorsil Huile 10634, by the company General Electric under the names SF1066 and SF1188, by the company SWS Silicones under the name Silicone Copolymer F 754, by the company Amerchol under the name Silsoft Beauty Aid SL, by the company Shin-Etsu under the name KF 351, by the company Wacker under the name Belsil DMC 6038, by the company Siltech under the names Silwax WD-C, Silwax WD-B, Silwax WD-IS, Silwax WSL, Silwax DCA 100 and Siltech Amine 65, by the company Fanning Corporation under the names Fancorsil SLA and Fancorsil LIM1, and by the company Phoenix under the name Pecosil.

These silicones are described, for example, in U.S. Pat. Nos. 5,070,171; 5,149,765; 5,093,452; and 5,091,493.

According to at least one embodiment, the polyoxyalkylenated silicones corresponding to formula (VII) or (VIII) are used. For example, these formulae correspond to at least one of, and according to at least one embodiment, all of the following conditions:
c is equal to 2 or 3;
R₁ is a methyl radical;
R₅ is chosen from a methyl radical, a C₁₂-C₂₂ acyl radical, and CO(CH₂)_{d}COOM;
a ranges from 2 to 25, and further, for example, from 2 to 15;
b is equal to 0;
n ranges from 0 to 100; and
p ranges from 1 to 20.

According to another embodiment, the polyoxyalkylenated silicones may be chosen from the silicones of formula (X) below:

([Z(R₂SiO)_{q}R′₂SiZO][(CₙH₂ₙO)ᵣ])ₛ (X)

wherein:
R₂ and R′₂, which may be identical or different, are C₁-C₃₀ monovalent hydrocarbon-based radicals;
n is an integer ranging from 2 to 4;
q is a number greater than or equal to 4, such as from 4 to 200, and even further, for example, from 4 to 100;
r is a number greater than or equal to 4, such as from 4 to 200, and even further, for example, from 5 to 100;
s is a number greater than or equal to 4, such as from 4 to 1000, and even further, for example, from 5 to 300;
Z is a divalent organic group which is linked to the adjacent silicon atom via a carbon-silicon bond and to the polyoxyalkylene (CₙH₂ₙO) block via an oxygen atom;
the average molecular weight of each siloxane block ranges from 400 to 10,000, that of each polyoxyalkylene block ranging from 300 to 10,000;
the siloxane blocks are present in an amount ranging from 10% to 95% by weight of the block copolymer;
it being possible for the number-average molecular weight of the block copolymer to range from 2,500 to 1,000,000, for example, from 3,000 to 200,000, and even further, for example, from 6,000 to 100,000.

R₂ and R′₂ may be chosen from the group comprising linear or branched alkyl radicals, for instance methyl, ethyl, propyl, butyl, pentyl, hexyl, octyl, decyl or dodecyl radicals, aryl radicals, for instance phenyl or naphthyl, aralkyl or alkylaryl radicals, for instance benzyl or phenylethyl, and tolyl and xylyl radicals.

Z is —R″—, —R″—CO—, R″—NHCO—, R″—NH—CO—NH—R′″— or —R″—OCONH—R′″—NHCO—, where R″ is a linear or branched C₁-C₆ divalent alkylene, for instance linear or branched ethylene, propylene or butylene, and R′″ is a divalent alkylene group or a divalent arylene group such as —C₆H₄—, —C₆H₄—C₆H₄—, —C₆H₄—CH₂—C₆H₄— or —C₆H₄—C(CH₃)₂C₆H₄—.

According to at least one embodiment, Z is a divalent alkylene radical, such as the —C₃H₆— radical or the C₄H₈ radical, which may be linear or branched.

The preparation of the block copolymers is described in European Application EP 0 492 657 A1, the disclosure of which is incorporated herein by reference.

Such products are, for example, sold under the name Silicone Fluid FZ-2172 by the company OSI.

The silicones used can be in the form of aqueous solutions, i.e. in solubilized form, or optionally in the form of dispersions or microdispersions, or of aqueous emulsions.

The silicone(s) that can be used in the cosmetic composition can also be silicone gums.

The silicone gums that can be used in the cosmetic composition include, for example, polydiorganosiloxanes having high weight-average molecular weights ranging from 200,000 to 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane, tridecanes, and mixtures thereof.

Non-limiting mention may be made of the following products:
polydimethylsiloxane,
polydimethylsiloxane/methylvinylsiloxane gums,
polydimethylsiloxane/diphenylmethylsiloxane,
polydimethylsiloxane/phenylmethylsiloxane,
polydimethylsiloxane/diphenylsiloxane/methylvinylsiloxane.

In at least one embodiment, products that can be used in accordance with the present disclosure are mixtures such as:
mixtures formed from a polydimethylsiloxane hydroxylated at the end of the chain (called dimethiconol according to the nomenclature of the CTFA dictionary) and from a cyclic polydimethylsiloxane (called cyclomethicone according to the nomenclature of the CTFA dictionary), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a molecular weight of 500,000, solubilized in the oil SF 1202 Silicone Fluid corresponding to decamethylcyclopentasiloxane;
mixtures of two PDMSs of different viscosities, and more particularly of a PDMS gum and of a PDMS oil, such as the product SF 1236 from the company General Electric. The product SF 1236 is the mixture of an SE 30 gum defined above having a viscosity of 20 m²/s and of an SF 96 oil having a viscosity of 5×10⁻⁶ m²/s. According to at least one embodiment, this product comprises 15% of SE 30 gum and 85% of an SF 96 oil.

The silicone(s) that can be used in the cosmetic composition can also be amino silicones.

As disclosed herein, the term “amino silicone” is understood to mean any silicone comprising at least one primary, secondary or tertiary function or a quaternary ammonium group.

The amino silicones that may be used in the cosmetic composition as disclosed herein, may be chosen from:

(a) the compounds corresponding to formula (XI) below:

(R¹)ₐ(T)₃₋ₐ-Si[OSi(T)₂]ₙ—[OSi(T)_{b}(R¹)_{2-b}]ₘ—OSi(T)₃₋ₐ-(R¹)ₐ (XI)

wherein:

T is a hydrogen atom, or a phenyl, hydroxyl (—OH), C₁-C₈ alkyl, such as methyl, or C₁-C₈ alkoxy, for example methoxy, radical;

a denotes the number 0 or an integer from 1 to 3, and according to at least one embodiment is 0;

b denotes 0 or 1, and according to at least one embodiment is 1;

m and n are numbers such that the sum (n+m) may range from 1 to 2,000, such as from 50 to 150, it being possible for n to denote a number from 0 to 1,999, such as from 49 to 149, and it being possible for m to denote a number from 1 to 2,000, for instance from 1 to 10;

R₁ is a monovalent radical of formula —C_{q}H_{2q}L in which q is a number from 2 to 8 and L is an optionally quaternized amino group chosen from the compounds:
—N(R²)—CH₂—CH₂—N(R²)₂;
—N(R²)₂; —N⁺(R²)₃—;
N⁺(R²)(H)₂Q-;
N⁺(R²)₂HQ-;
—N(R²)—CH₂—CH₂—N(R²)(H)₂O—;
   wherein R² is chosen from a hydrogen atom, a phenyl, a benzyl, or a monovalent saturated hydrocarbon-based radical, for example a C₁-C₂₀ alkyl radical, and Q- is a halide ion, such as, for example, fluoride, chloride, bromide or iodide.

For instance, the amino silicones corresponding to the definition of formula (XI) are chosen from the compounds corresponding to the formula (XII) below:

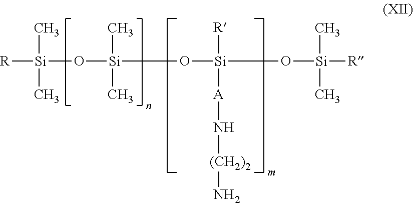

wherein R, R′ and R″, which may be identical or different, are C₁-C₄ alkyl radicals, for instance CH₃; a C₁-C₄ alkoxy radical, for example methoxy; or OH; A is a C₃-C₈, such as C₃-C₆, linear or branched alkylene radical; m and n are integers which depend on the molecular weight and the sum of which ranges from 1 to 2,000.

According to one embodiment, R, R′ and R″, which may be identical or different, are C₁-C₄ alkyl or hydroxyl radicals, A is a C₃ alkylene radical, and m and n are such that the weight-average molecular mass of the compound ranges from 5,000 to 500,000. The compounds of this type are referred to, in the CTFA dictionary, as “amodimethicone”.

According to another embodiment, R, R′ and R″, which may be identical or different, are C₁-C₄ alkoxy or hydroxyl radicals, at least one of the radicals R or R″ is an alkoxy radical and A is a C₃ alkylene radical. The hydroxyl/alkoxy molar ratio can range from 0.2/1 to 0.4/1, and according to at least one embodiment is equal to 0.3/1. Moreover, m and n are such that the weight-average molecular mass of the compound ranges from 2,000 to 10⁶. For instance, n ranges from 0 to 999 and m ranges from 1 to 1,000, wherein the sum of n and m ranges from 1 to 1000.

In this category of compounds, non-limiting mention may be made, inter alia, of the product Belsil®ADM 652 sold by Wacker.

According to another embodiment, R and R″, which are different, are chosen from C₁-C₄ alkoxy radicals and hydroxyl radicals, at least one of the radicals R and R″ is an alkoxy radical, R′ is a methyl radical and A is a C₃ alkylene radical. The hydroxyl/alkoxy molar ratio can range from 1/0.8 to 1/1.1, and according to at least one embodiment is equal to 1/0.95. Moreover, m and n are such that the weight-average molecular mass of the compound ranges from 2,000 to 200,000. For instance, n ranges from 0 to 999 and m ranges from 1 to 1,000, wherein the sum of n and m ranges from 1 to 1,000.

For instance, non-limiting mention may be made of the product Fluid WRF® 1300 sold by Wacker. [077]. According to a further embodiment, R and R″ are hydroxyl radicals, R′ is a methyl radical and A is a C₄-C₅, such as C₄, alkylene radical. Moreover, m and n are such that the weight-average molecular mass of the compound ranges from 2,000 to 10⁶. For instance, n ranges from 0 to 1,999 and m ranges from 1 and 2,000, the sum of n and m ranges from 1 to 2,000.

For example, non-limiting mention may be made of the product sold under the name DC28299 by Dow Corning.

It should be noted that the molecular mass of these silicones is determined by gel permeation chromatography (ambient temperature, polystyrene standard; p styragem columns; eluent THF; flow rate of 1 mm/m; 200 μl of a solution at 0.5% by weight of silicone in THF are injected and the detection is carried out by refractometry and UV-metry).

A product corresponding to the definition of formula (XI) includes, for example, the polymer referred to, in the CTFA dictionary, as “trimethylsilylamodimethicone”, corresponding to formula (XIII) below:

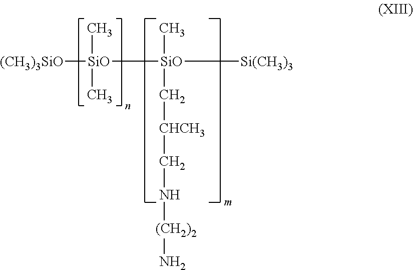

wherein n and m are defined above, in accordance with formula (XI).

Such compounds are described, for example, in EP 095 238; a compound of formula (XIII) is, for example, sold under the name Q2-8220 by the company OSI;

(b) the compounds corresponding to formula (XIV) below:

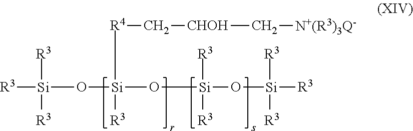

wherein:

R³ is a C₁-C₁₈ monovalent hydrocarbon-based radical, such as a C₁-C₁₈ alkyl radical or a C₂-C₁₈ alkenyl radical, for example methyl;

R⁴ is a divalent hydrocarbon-based radical, such as a C₁-C₆₁ alkylene radical or a C₁-C₁₈ divalent alkyleneoxy radical, for example C₁-C₈;

Q⁻ is a halide ion, such as chloride;

r represents an average statistical value from 2 to 20, for instance from 2 to 8;

s represents an average statistical value from 20 to 200, for instance from 20 to 50.

Such compounds are described, for example, in U.S. Pat. No. 4,185,087.

A compound which falls within this category is that sold by the company Union Carbide under the name “Ucar Silicone ALE 56”;

c) quaternary ammonium silicones of formula (XV):

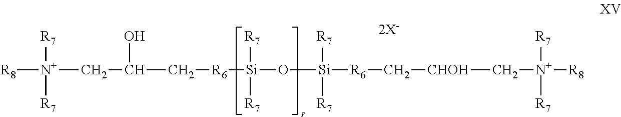

wherein:

R₇, which may be identical or different, is a monovalent hydrocarbon-based radical containing from 1 to 18 carbon atoms, for instance, a C₁-C₁₈ alkyl radical, a C₂-C₁₈ alkenyl radical or a ring comprising 5 or 6 carbon atoms, for example methyl;

R₆ is a divalent hydrocarbon-based radical, for instance, a C₁-C₁₈ alkylene radical or a C₁-C₁₈ divalent alkyleneoxy radical, for example C₁-C₈, linked to the Si via an SiC bond;

R₈, which may be identical or different, is chosen from a hydrogen atom, a monovalent hydrocarbon-based radical containing from 1 to 18 carbon atoms, such as a C₁-C₁₈ alkyl radical, a C₂-C₁₈ alkenyl radical or a radical —R₆—NHCOR₇;

X— is an anion such as a halide ion, e.g., chloride, or an organic acid salt (acetate, etc.);

r represents an average statistical value from 2 to 200, such as from 5 to 100;

These silicones are, for example, described in application EP-A-0 530 974;

d) amino silicones of formula (XVI):

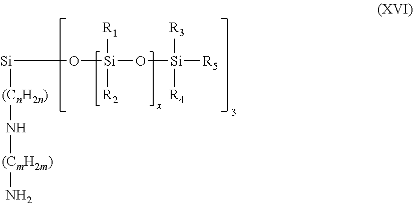

wherein:
R₁, R₂, R₃ and R₄, which may be identical or different, are chosen from C₁-C₄ alkyl radicals and phenyl groups;
R₅ is chosen from a C₁-C₄ alkyl radical and a hydroxyl group;
n is an integer ranging from 1 to 5;
m is an integer ranging from 1 to 5;
   and wherein x is chosen such that the amine index ranges from 0.01 to 1 meq/g.

As disclosed herein, the silicones that may be used include, but are not limited to, polysiloxanes comprising amino groups, such as amodimethicones or trimethylsilylamodimethicones (CTFA, 4th edition, 1997), and even further, for example, silicones comprising quaternary ammonium groups.

When these compounds are used, one embodiment relates to their joint use with cationic and/or nonionic surface agents.

By way of example, use may be made of the product sold under the name “Emulsion Cationique DC 929” by the company Dow Corning, which comprises, in addition to the amodimethicone, a cationic surface agent comprising a mixture of products corresponding to the formula:

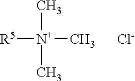

wherein R⁵ is a C₁₄-C₂₂ alkenyl and/or alkyl radical derived from tallow fatty acids, and known under the CTFA name “tallowtrimonium chloride”, in combination with a nonionic surface agent of formula:

C₉H₁₉—C₆H₄—(OC₂H₄)₁₀—OH,

known under the CTFA name “Nonoxynol 10”.

Use may also be made, for example, of the product sold under the name “Emulsion Cationique DC 939” by the company Dow Corning, which comprises, in addition to the amodimethicone, a cationic surface agent which is trimethylcetylammonium chloride and a nonionic surface agent of formula: C₁₃H₂₇—(OC₂H₄)₁₂—OH, known under the CTFA name “trideceth-12”.

Another commercial product that can be used according to the present disclosure is the product sold under the name “Dow Corning Q2 7224” by the company Dow Corning, comprising a combination of the trimethylsilylamodimethicone of formula (C) described above, a nonionic surface agent of formula: C₈H₁₇—C₆H₄—(OCH₂CH₂)₄₀—OH, known under the CTFA name “octoxynol-40”, a second nonionic surface agent of formula: C₁₂H₂₅—(OCH₂—CH₂)₆—OH, known under the CTFA name “isolaureth-6”, and propylene glycol.

The silicone(s) is (are) present in the composition in an amount ranging from 0.1% to 30%, for instance in an amount ranging from 0.2% to 20%, and even further, for example, in an amount ranging from 0.5% to 10% by weight, relative to the total weight of the composition.

The cosmetic composition according to the present disclosure may further comprise a non-silicone fatty substance such as plant, animal, mineral and synthetic oils, fatty alcohols, fatty acids and waxes.

The fatty substance(s) is (are) present in the composition in an amount ranging from 0.1% to 30%, for instance in an amount ranging from 0.2% to 20%, and even further, for example, in an amount ranging from 0.5% to 10% by weight, relative to the total weight of the composition.

For the purpose of the present disclosure, the term “fatty alcohol” is understood to mean any linear or branched, saturated or unsaturated pure fatty alcohol containing at least 8 carbon atoms. The fatty alcohol may be oxyalkylenated or glycerolated.

The fatty alcohol may have the structure R—OH, in which R is a linear or branched, saturated or unsaturated radical containing from 8 to 40 carbon atoms, such as from 8 to 30; according to at least one embodiment, R is a C₁₂-C₂₄ alkyl or C₁₂-C₂₄ alkenyl group. R can also be substituted with at least one hydroxyl group.

By way of example of fatty alcohols, non-limiting mention may be made of lauryl alcohol, cetyl alcohol, dodecyl alcohol, decyl alcohol, stearyl alcohol, oleyl alcohol, behenyl alcohol, linoleyl alcohol, undecylenyl alcohol, palmitoleyl alcohol, arachidonoyl alcohol, erucyl alcohol, and mixtures thereof.

The fatty alcohol may be present as a mixture of fatty alcohols, which means that several species of fatty alcohols can coexist, in the form of a mixture, in a commercial product.

By way of a mixture of fatty alcohols, non-limiting mention may be made of cetylstearyl alcohol or cetearyl alcohol.

According to at least one embodiment, the nonoxyalkylenated fatty alcohol is solid or pasty at a temperature of 25° C. As disclosed herein, the expression “fatty alcohol solid or pasty at 25° C.” is understood to mean a fatty alcohol having a viscosity, measured with a rheometer with a shear rate of 1 s⁻¹, of greater than or equal to 1 Pa·s.

According to another embodiment, the fatty alcohols used in the cosmetic composition according to the present disclosure are cetyl alcohol and cetearyl alcohol.

For the purpose of the present disclosure, the term “fatty acids” is understood to mean any linear or branched, saturated or unsaturated pure carboxylic acid containing at least 8 carbon atoms. By way of examples of a fatty acid, non-limiting mention may be made of lauric acid and oleic acid.

The cosmetic composition can comprise at least one fixing polymer other than carrageenans.

For the purpose of the present disclosure, the term “fixing polymer” is understood to mean any polymer that makes it possible to confer a given shape or to hold a given shape or hairstyle.

The fixing polymers that can be used in the cosmetic composition according to the present disclosure may be chosen from cationic, anionic, amphoteric or nonionic polymers and mixtures thereof other than the fixing material described above.

For the purpose of the present disclosure, the term “cationic polymer” is understood to mean any polymer comprising cationic groups and/or groups that can be ionized to cationic groups.

The cationic fixing polymers that can be used in the cosmetic composition according to the present disclosure can be chosen from polymers comprising primary, secondary, tertiary and/or quaternary amine groups that are part of the polymer chain or directly linked thereto, and having a number-average molecular mass that ranges from 500 to 5,000,000, and according to at least one embodiment, from 1,000 to 3,000,000.

Among these polymers, non-limiting mention may be made of the following cationic polymers:

(1) homopolymers or copolymers of acrylic or methacrylic esters or amides, with amino functions, comprising at least one of the units of the formulae below:

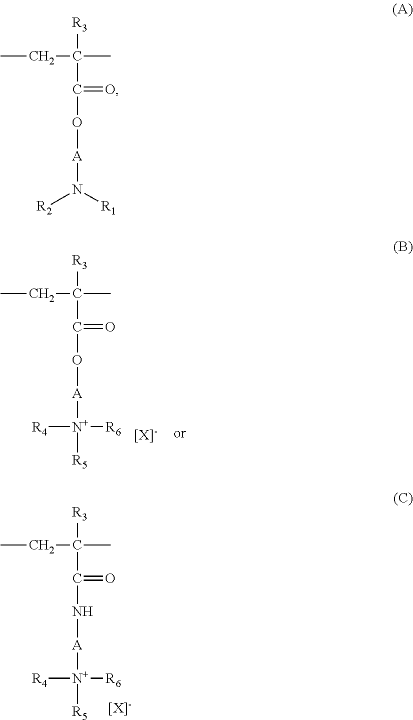

wherein:

R₁ and R₂, which may be identical or different, are chosen from a hydrogen atom and an alkyl group containing from 1 to 6 carbon atoms;

R₃ is a hydrogen atom or a CH₃ group;

A is a linear or branched alkyl group containing from 1 to 6 carbon atoms or a hydroxyalkyl group containing from 1 to 4 carbon atoms;

R₄, R₅ and R₆, which may be identical or different, are alkyl groups containing from 1 to 18 carbon atoms or a benzyl group; and

X denotes a methosulphate anion or a halide such as chloride or bromide.

The copolymers of family (1) also contain at least one unit derived from comonomers which can be chosen from the family of acrylamides, methacrylamides, diacetone acrylamides, acrylamides and methacrylamides substituted on the nitrogen with lower (C₁-C₄) alkyl groups, groups derived from acrylic or methacrylic acids or their esters, vinyllactams, such as vinylpyrrolidone or vinylcaprolactam, or vinyl esters.

Thus, among these copolymers of family (1), non-limiting mention may be made of:
copolymers of acrylamide and of dimethylaminoethyl methacrylate quaternized with dimethyl sulphate or with a dimethyl halide, such as that sold under the name Hercofloc® by the company Hercules,
copolymers of acrylamide and of methacryloyloxyethyltrimethylammonium chloride, described, for example, in patent application EP-A-080976 and sold under the name Bina Quat P 100 by the company Ciba Geigy,
copolymers of acrylamide and of methacryloyloxyethyltrimethylammonium methosulphate, such as that sold under the name Reten by the company Hercules,
vinylpyrrolidone/dialkylaminoalkyl acrylate or methacrylate copolymers, which may or may not be quaternized, such as the products sold under the name “Gafquat®” by the company ISP, such as, for example, “Gafquat® 734” or “Gafquat® 755”, or else the products called “Copolymer) 845, 958 and 937”. These polymers are described in detail in French Patent Nos. 2 077 143 and 2 393 573,
dimethylaminoethyl methacrylate/vinylcaprolactam/vinylpyrrolidone terpolymers, such as the product sold under the name Gaffix® VC 713 by the company ISP, and
vinylpyrrolidone/quaternized dimethylaminopropylmethacrylamide copolymers, such as, for example, the product sold under the name “Gafquat® HS 100” by the company ISP;

(2) quaternary copolymers of vinylpyrrolidone and of vinylimidazole;

(3) chitosans or their salts; salts that can be used are, for example, chitosan acetate, lactate, glutamate, gluconate and pyrrolidonecarboxylate.

Among these compounds, non-limiting mention may be made of the chitosan having a degree of deacetylation of 90.5% by weight, sold under the name Kytan Brut Standard by the company Aber Technologies, and the chitosan pyrrolidonecarboxylate sold under the name Kytamer® PC by the company Amerchol.

The commercialized products corresponding to this definition include, for example, the products sold under the name “Celquat L 200” and “Celquat H 100” by the company National Starch.

The anionic fixing polymers generally used are polymers comprising groups derived from carboxylic acid, sulphonic acid or phosphoric acid and have a number-average molecular mass that ranges from 500 to 5,000,000.

The carboxylic groups are provided by unsaturated mono- or dicarboxylic acid monomers such as those corresponding to the formula:

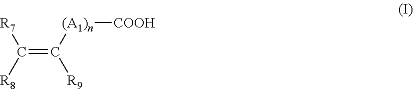

wherein:

n is an integer from 0 to 10;

A₁ is a methylene group optionally linked to the carbon atom of the unsaturated group or to the neighboring methylene group when n is greater than 1, via a heteroatom such as oxygen or sulphur;

R₇ is chosen from a hydrogen atom, a phenyl group, and a benzyl group;

R₈ is chosen from a hydrogen atom, a lower alkyl group, and a carboxyl group; and

R₉ is chosen from a hydrogen atom, a lower alkyl group, a —CH₂—COOH group, a phenyl group, or a benzyl group.

In the above-mentioned formula, a lower alkyl group, according to at least one embodiment, is a group containing 1 to 4 carbon atoms, such as methyl and ethyl groups.

The anionic fixing polymers with carboxylic groups, that may be used according to the present disclosure, include but are not limited to:

A) Homopolymers or copolymers of acrylic or methacrylic acid or their salts, for instance the products sold under the names Versicol® E or K by the company Allied Colloid, and Ultrahold® by the company BASF, the acrylic acid and acrylamide copolymers sold in the form of their sodium salts under the names Reten 421, 423 or 425 by the company Hercules, and sodium salts of polyhydroxycarboxylic acids.

B) Copolymers of acrylic or methacrylic acid with a monoethylenic monomer such as ethylene, styrene, vinyl esters, esters of acrylic or methacrylic acid, optionally grafted onto a polyalkylene glycol such as polyethylene glycol, and optionally crosslinked. Such polymers are described, for example, in French Patent No. 1 222 944 and German Application No. 2 330 956, copolymers of this type comprising, in their chain, an optionally N-alkylated and/or hydroxyalkylated acrylamide unit, as described for instance in Luxemburg Patent Application Nos. 75370 and 75371 or proposed under the name Quadramer by the company American Cyanamid. Non-limiting mention may also be made of methacrylic acid/ethyl acrylate/tert-butyl acrylate terpolymers such as the product sold under the name Luvimer® 100 P by the company BASF.

Non-limiting mention may also be made of methacrylic acid/acrylic acid/ethyl acrylate/methyl methacrylate copolymers as an aqueous dispersion, sold under the name Amerhold® DR 25 by the company Amerchol.

C) Copolymers of crotonic acid, such as those comprising, in their chain, vinyl acetate or propionate units, and optionally other monomers such as allyl or methallyl esters, vinyl ether or vinyl ester of a linear or branched, saturated carboxylic acid comprising a long hydrocarbon-based chain, such as those containing at least 5 carbon atoms, it being possible for these polymers to be optionally grafted or crosslinked, or else another monomer of a vinyl, allyl or methallyl ester of an α- or β-cyclic carboxylic acid. Such polymers are described, inter alia, in French Patent Nos. 1 222 944; 1 580 545; 2 265 782; 2 265 781; 1 564 110; and 2 439 798. Commercial products which fall into this class are the resins 28-29-30, 26-13-14 and 28-13-10 sold by the company National Starch.

D) Copolymers of C₄-C₈ monounsaturated carboxylic acids or anhydrides, chosen from:
copolymers comprising (i) at least one maleic, fumaric or itaconic acid or anhydride; and (ii) at least one monomer chosen from vinyl esters, vinyl ethers, vinyl halides, phenylvinyl derivatives, and acrylic acid and its esters, the anhydride functions of these copolymers being optionally monoesterified or monoamidated. Such polymers are described, for example, in U.S. Pat. Nos. 2,047,398; 2,723,248 and 2,102,113; and GB Patent No. 839 805. Commercial products include, for instance, those sold under the names Gantrez® AN or ES by the company ISP;
copolymers comprising (i) at least one maleic, citraconic or itaconic anhydride unit; and (ii) at least one monomer chosen from allyl or methallyl esters optionally comprising at least one acrylamide, methacrylamide, α-olefin, acrylic or methacrylic ester, acrylic or methacrylic acid or vinylpyrrolidone groups in their chain,

the anhydride functions of these copolymers being optionally monoesterified or monoamidated.

These polymers are, for example, described in French Patent Nos. 2 350 384 and 2 357 241 by the applicant.

E) Polyacrylamides comprising carboxylate groups.

The homopolymers and copolymers comprising sulphonic groups include, but are not limited to, polymers comprising vinylsulphonic, styrenesulphonic, naphthalenesulphonic or acrylamidoalkylsulphonic units.

These polymers can, for instance, be chosen from:
the salts of polyvinylsulphonic acid having a molecular mass ranging from 1,000 to 100,000, and also the copolymers with an unsaturated comonomer such as acrylic or methacrylic acids and their esters, and also acrylamide or its derivatives, vinyl ethers and vinylpyrrolidone;
the salts of polystyrenesulphonic acid such as the sodium salts sold, for example, under the names Flexan® 500 and Flexan® 130 by National Starch. These compounds are described in French Patent No. FR 2 198 719;
the salts of polyacrylamidesulphonic acids such as those mentioned in U.S. Pat. No. 4,128,631, and according to at least one embodiment, the polyacrylamidoethylpropanesulphonic acid sold under the name Cosmedia Polymer HSP 1180 by Henkel.

For example, the anionic fixing polymers are chosen from acrylic acid copolymers such as the acrylic acid/ethyl acrylate/N-tert-butylacrylamide terpolymers including those sold under the name Ultrahold® Strong by the company BASF, copolymers derived from crotonic acid, such as the vinyl acetate/vinyl tert-butyl benzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids or anhydrides with vinyl esters, vinyl ethers, vinyl halides, phenylvinyl derivatives, acrylic acid and its esters, such as the methyl vinyl ether/monoesterified maleic anhydride copolymers sold, for example, under the name Gantrez® by the company ISP, copolymers of methacrylic acid and of methyl methacrylate sold under the name Eudragit® L by the company Rohm Pharma, copolymers of methacrylic acid and of ethyl acrylate sold under the name Luvimer) MAEX or MAE by the company BASF and the vinyl acetate/crotonic acid copolymers sold, for example, under the name Luviset CA 66 by the company BASF and the vinyl acetate/crotonic acid copolymers grafted with polyethylene glycol, sold under the name Aristoflex® A by the company BASF.

Among the anionic fixing polymers mentioned above, use may be made, in the context of the present disclosure, of the methyl vinyl ether/monoesterified maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butyl benzoate/crotonic acid terpolymers and the crotonic acid/vinyl acetate/vinyl neododecanoate terpolymers sold under the name Resin 28-29-30 by the company National Starch, and the copolymers of methacrylic acid and of ethyl acrylate sold under the name Luvimer® MAEX or MAE by the company BASF.

The amphoteric fixing polymers that can be used in accordance with the present disclosure may be chosen from polymers comprising units B and C randomly distributed in the polymer chain, where B is a unit derived from a monomer comprising at least one basic nitrogen atom and C is a unit derived from an acid monomer comprising at least one carboxylic or sulphonic groups, or else B and C can denote groups derived from zwitterionic monomers of carboxybetaines or of sulphobetaines.

B and C can also denote a cationic polymer chain comprising primary, secondary, tertiary or quaternary amine groups, in which at least one of the amine groups bears a carboxylic or sulphonic group linked via a hydrocarbon-based group, or else B and C are part of a chain of a polymer comprising an ethylene-α,β-dicarboxylic unit, one of the carboxylic groups of which has been brought to react with a polyamine comprising at least one primary or secondary amine group.

The amphoteric fixing polymers corresponding to the definition given above that may be used, as disclosed herein, may be chosen from the following polymers:

(1) Copolymers comprising acidic vinyl units and basic vinyl units, such as those resulting from the copolymerization of a monomer derived from a vinyl compound bearing a carboxylic group such as, for example, acrylic acid, methacrylic acid, maleic acid or alpha-chloracrylic acid, and of a basic monomer derived from a substituted vinyl compound containing at least one basic atom, such as, for example, dialkylaminoalkyl methacrylates and acrylates, dialkylaminoalkylmethacrylamide and acrylamide. Such compounds are described in U.S. Pat. No. 3,836,537.

(2) Polymers comprising units deriving:
a) from at least one monomer chosen from acrylamides or methacrylamides substituted on the nitrogen atom with an alkyl group;
b) from at least one acidic comonomer comprising at least one reactive carboxylic groups; and
c) from at least one basic comonomer, such as esters comprising primary, secondary, tertiary and quaternary amine substituents of acrylic and methacrylic acids, and the quaternization product of dimethylaminoethyl methacrylate with dimethyl or diethyl sulphate.

The N-substituted acrylamides or methacrylamides according to the present disclosure include, but are not limited to, the compounds in which the alkyl groups contain from 2 to 12 carbon atoms, and further, for example, N-ethylacrylamide, N-tert-butylacrylamide, N-tert-octylacrylamide, N-octylacrylamide, N-decylacrylamide and N-dodecylacrylamide, and also the corresponding methacrylamides.

The acidic comonomers are chosen, for instance, from acrylic acid, methacrylic acid, crotonic acid, itaconic acid, maleic acid and fumaric acid, and also the monoalkyl esters having 1 to 4 carbon atoms of maleic or fumaric acids or anhydrides.

The basic comonomers that may be used include, but are not limited to, aminoethyl methacrylate, butylaminoethyl methacrylate, N,N′-dimethylaminoethyl methacrylate and N-tert-butylaminoethyl methacrylate.

For instance, as disclosed herein, use may be made of the copolymers whose CTFA name (4th Ed., 1991) is octylacrylamide/acrylates/butylaminoethyl methacrylate copolymer, such as the products sold under the name Amphomer® or Lovocryl® 47 by the company National Starch.

(3) Partially or completely acylated and crosslinked polyaminoamides deriving from polyaminoamides of formula:

CO—R₁₀CO—Z (XVII)

wherein:

R₁₀ is a divalent group derived from a saturated dicarboxylic acid, from an aliphatic mono- or dicarboxylic acid comprising an ethylenic double bond, from an ester of a lower alkanol having 1 to 6 carbon atoms of these acids, or from a group derived from the addition of any one of said acids with a bisprimary or bissecondary amine; and

Z is a group derived from a bisprimary, monosecondary or bissecondary poly-alkylenepolyamine, and represents, for example:

a) in the amount ranging from 60 to 100 mol %, the group

—NH(CH₂)ₓ—NHₚ (XVIII)

wherein x=2 and p=2 or 3, or else x=3 and p=2,
this group deriving from diethylenetriamine, triethylenetetramine or dipropylenetriamine;

b) in the amount ranging from 0 to 40 mol %, the above group (XVIII), in which x=2 and p=1 and which derives from ethylenediamine, or the group deriving from piperazine:

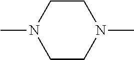

c) in the amount ranging from 0 to 20 mol %, the group —NH—(CH₂)₆—NH— deriving from hexamethylenediamine,

wherein these polyaminoamides are crosslinked by addition reaction of a bifunctional crosslinking agent chosen from epihalohydrins, diepoxides, dianhydrides and bisunsaturated derivatives, using from 0.025 to 0.35 mol of crosslinking agent per amine group of the polyaminoamide, and acylated by the action of acrylic acid, chloroacetic acid or an alkane sultone, or their salts.

The saturated carboxylic acids can be chosen, for example, from acids having 6 to 10 carbon atoms, such as adipic acid, 2,2,4-trimethyladipic acid, 2,4,4-trimethyladipic acid or terephthalic acid, or acids comprising an ethylenic double bond, such as, for example, acrylic acid, methacrylic acid or itaconic acid.

The alkane sultones used in the acylation include, for instance, propane sultone or butane sultone, and the salts of the acylating agents, such as the sodium or potassium salts.

(4) Polymers comprising zwitterionic units of formula:

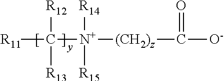

wherein R₁₁ is a polymerizable unsaturated group, such as an acrylate, methacrylate, acrylamide or methacrylamide group, y and z are integers ranging from 1 to 3, R₁₂ and R₁₃ are chosen from a hydrogen atom or a methyl, ethyl or propyl group, and R₁₄ and R₁₅ are chosen from a hydrogen atom or an alkyl group such that the sum of the carbon atoms in R₁₄ and R₁₅ does not exceed 10.

The polymers comprising such units can also comprise units derived from non-zwitterionic monomers, such as dimethylaminoethyl acrylate, dimethylaminoethyl methacrylate, diethylaminoethyl acrylate, diethylaminoethyl methacrylate, alkyl acrylates, alkyl methacrylates, acrylamides, methacrylamides or vinyl acetate.

By way of example, non-limiting mention may be made of methyl methacrylate/methyl dimethylcarboxymethylammonioethylmethacrylate copolymers, such as the product sold under the name Diaformer Z301 by the company Sandoz.

(5) Polymers derived from chitosan comprising monomeric units corresponding to the formulae below:

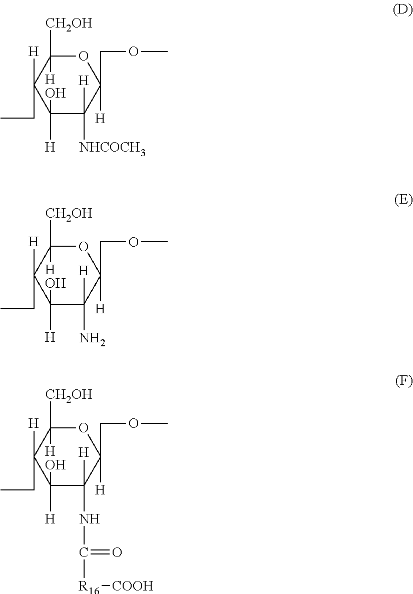

the unit (D) being present in an amount ranging from 0 to 30%, the unit (E) in an amount ranging from 5 to 50% and the unit (F) in an amount ranging from 30 to 90%, it being understood that, in this unit (F), R₁₆ represents a group of formula:

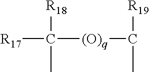

wherein, if q=0, R₁₇, R₁₈ and R₁₉, which may be identical or different, are each chosen from hydrogen atom, a methyl, hydroxyl, acetoxy or amino residue, a monoalkylamine residue or a dialkylamine residue which are optionally interrupted with at least one nitrogen atom and/or optionally substituted with at least one amino, hydroxyl, carboxyl, alkylthio or sulphonic groups, or an alkylthio residue, the alkyl group of which bears an amino residue, at least one of the groups R₁₇, R₁₈ and R₁₉ being, in this case, a hydrogen atom; or, if q=1, R₁₇, R₁₈ and R₁₉ each represent a hydrogen atom, and also the salts formed by these compounds with bases or acids.

(6) Polymers comprising units corresponding to formula (XIX) are, for example, described in French Patent No. 1,400,366:

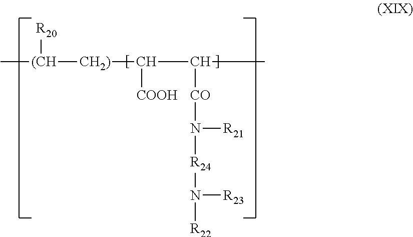

in which R₂₀ is chosen from a hydrogen atom, a CH₃O, CH₃CH₂O group, and a phenyl group, R₂₁ is chosen from a hydrogen atom and a lower alkyl group such as methyl or ethyl, R₂₂ is chosen from a hydrogen atom and a C₁-C₆ lower alkyl group such as methyl or ethyl, R₂₃ is chosen from a C₁-C₆ lower alkyl group, such as methyl or ethyl, and a group corresponding to the formula: —R₂₄—N(R₂₂)₂, where R₂₄ is a —CH₂—CH₂—, —CH₂CH₂—CH₂— or —CH₂—CH(CH₃)— group, and where R₂₂ is defined as mentioned above.

(7) Polymers derived from the N-carboxyalkylation of chitosan, such as N-carboxymethylchitosan or N-carboxybutylchitosan, sold under the name “Evalsan” by the company Jan Dekker.

(8) Amphoteric polymers of the -D-X-D-X type chosen from:

a) polymers obtained by the action of chloroacetic acid or sodium chloroacetate on compounds comprising at least one unit of formula:

-D-X-D-X-D- (XX)

where D denotes a group

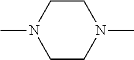

and X denotes the symbol E or E′, E or E′, which may be identical or different, denoting a divalent group which is a straight- or branched-chain alkylene group containing up to 7 carbon atoms in the main chain, which is or is not substituted with hydroxyl groups and which can additionally comprise oxygen, nitrogen or sulphur atoms, 1 to 3 aromatic and/or heterocyclic rings; the oxygen, nitrogen and sulphur atoms being present in the form of ether, thioether, sulphoxide, sulphone, sulphonium, alkylamine, alkenylamine, hydroxyl, benzylamine, amine oxide, quaternary ammonium, amide, imide, alcohol, ester and/or urethane groups;

b) the polymers of formula:

-D-X-D-X— (XXI′)

where D denotes a group

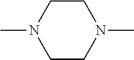

and X denotes the symbol E or E′ and at least once E′; E having the meaning indicated above and E′ being a divalent group which is a straight- or branched-chain alkylene group having up to 7 carbon atoms in the main chain, which is or is not substituted with at least one hydroxyl group and which comprises at least one nitrogen atom, the nitrogen atom being substituted with an alkyl chain optionally interrupted with an oxygen atom and necessarily comprising at least one carboxyl functions or at least one hydroxyl functions and betainized by a reaction with chloroacetic acid or sodium chloroacetate.

(9) (C₁-C₅)alkyl vinyl ether/maleic anhydride copolymers partially modified by semiamidation with an N,N-dialkylaminoalkylamine, such as N,N-dimethylamino-propylamine, or by semiesterification with an N,N-dialkylaminoalkanol. These copolymers may also comprise other vinyl comonomers such as vinylcaprolactam.

Among the amphoteric fixing polymers described above, according to at least one embodiment of the present disclosure, the amphoteric fixing polymers are those of family (3), such as copolymers whose CTFA name is octylacrylamide/acrylates/butylaminoethyl methacrylate copolymer, such as the products sold under the names Amphomer®, Amphomer® LV 71 or Lovocryl® 47 by the company National Starch, and those of family (4), such as the methyl methacrylate/methyl dimethylcarboxymethylammonioethyl methacrylate copolymers sold, for example, under the name Diaformer® Z301 by the company Sandoz.

The nonionic fixing polymers that can be used according to the present disclosure are chosen, for example, from:
polyalkyloxazolines;
vinyl acetate homopolymers;
vinyl acetate copolymers such as, for example, copolymers of vinyl acetate and of acrylic ester, copolymers of vinyl acetate and of ethylene, or copolymers of vinyl acetate and of maleic ester, for example of dibutyl maleate;
homopolymers and copolymers of acrylic esters, such as, for example, copolymers of alkyl acrylates and of alkyl methacrylates such as the products proposed by the company Rohm & Haas under the names Primal) AC-261 K and Eudragit® NE 30 D, by the company BASF under the name 8845, and by the company Hoechst under the name Appretan® N9212;
copolymers of acrylonitrile and of a nonionic monomer, chosen, for example, from butadiene and alkyl (meth)acrylates; non-limiting mention may be made of the products proposed under the name CJ 0601 B by the company Rohm & Haas;
styrene homopolymers;
styrene copolymers, for instance copolymers of styrene and of alkyl (meth)acrylate, such as the products Mowilith® LDM 6911, Mowilith® DM 611 and Mowilith® LDM 6070 proposed by the company Hoechst, the products Rhodopas® SD 215 and Rhodopas® DS 910 proposed by the company Rhodia Chimie; copolymers of styrene, of alkyl methacrylate and of alkyl acrylate; copolymers of styrene and of butadiene; or copolymers of styrene, of butadiene and of vinylpyridine;
polyamides;
vinyllactam homopolymers other than vinylpyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec VPC 55K65W by the company BASF, poly(vinylpyrrolidone/vinyl acetate) copolymers such as those sold under the name PVPVA® S630L by the company ISP, and Luviskol® VA 73, VA 64, VA 55, VA 37 and VA 28 by the company BASF; and poly(vinylpyrrolidone/vinyl acetate/vinyl propionate) terpolymers such as, for example, that sold under the name Luviskol® VAP 343 by the company BASF.

According to at least one embodiment, the alkyl groups of the nonionic polymers mentioned above have from 1 to 6 carbon atoms.

Use may also be made, as fixing polymers, of cationic, nonionic, anionic or amphoteric, silicone or nonsilicone, functionalized or nonfunctionalized polyurethanes, or mixtures thereof.

The polyurethanes used according to at least one embodiment, as disclosed herein, are those described in patent applications EP 0 751 162; EP 0 637 600; EP 0 648 485; and FR 2 743 297, of L'Oreal, and also in patent applications EP 0 656 021 and WO 94/03510 by the company BASF, and further described in EP 0 619 111 by the company National Starch.

For example, among the polyurethanes that are suitable in the present disclosure, non-limiting mention may be made of the products sold under the names Luviset Pur® and Luviset® Si Pur by the company BASF.

The additional fixing polymer(s) is (are) present in the cosmetic composition according to the present disclosure in an amount ranging from 0.01% to 20% by weight, such as from 0.05% to 15% by weight, and even further, for example, from 0.1% to 10% by weight, relative to the total weight of the cosmetic composition.

The composition according to the present disclosure can also contain surfactants.

The ionic surfactant(s) used in the cosmetic composition can be cationic surfactants.

By way of example of cationic surfactants that can be used in the cosmetic composition, non-limiting mention may be made, for instance, of optionally polyoxyalkylenated primary, secondary or tertiary fatty amine salts, quaternary ammonium salts, and mixtures thereof.

By way of quaternary ammonium salts, non-limiting mention may be made, for example, of:
those which have formula (I) below:

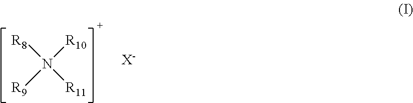

in which the radicals R₈ to R₁₁, which may be identical or different, are chosen from linear or branched aliphatic radicals containing from 1 to 30 carbon atoms, and aromatic radicals such as aryl or alkylaryl. The aliphatic radicals can comprise heteroatoms such as, for example, oxygen, nitrogen or sulphur, and halogens.

The aliphatic radicals are, for example, chosen from alkyl, alkoxy, polyoxy(C₂-C₆)alkylene, alkylamide, (C₁₂C₂₂)alkylamido(C₂-C₆)alkyl, (C₁₂-C₂₂)alkyl acetate and hydroxyalkyl radicals, containing from 1 to 30 carbon atoms; X is an anion chosen from halides, phosphates, acetates, lactates, (C₂-C₆)alkyl sulphates, alkyl sulphonates and alkylaryl sulphonates;
imidazoline quaternary ammonium salts, for instance those of formula (II) below:

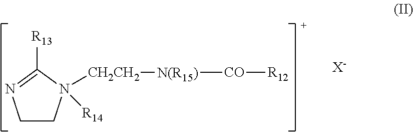

in which R₁₂ represents an alkenyl or alkyl radical containing from 8 to 30 carbon atoms, for example tallow fatty acid derivatives, R₁₃ is chosen from a hydrogen atom, a C₁-C₄ alkyl radical, and an alkenyl or alkyl radical containing from 8 to 30 carbon atoms, R₁₄ is a C₁-C₄ alkyl radical, R₁₅ is chosen from a hydrogen atom and a C₁-C₄ alkyl radical, X— is an anion chosen from halides, phosphates, acetates, lactates, alkyl sulphates, alkyl sulphonates and alkylaryl sulphonates. According to at least one embodiment, R₁₂ and R₁₃ denote a mixture of alkenyl or alkyl radicals containing from 12 to 21 carbon atoms, for example tallow fatty acid derivatives, R₁₄ is a methyl radical, R₁₅ is a hydrogen atom. Such a product is, for example, sold under the name Rewoquat® W 75 by the company Rewo;
diquaternary ammonium salts of formula (III):

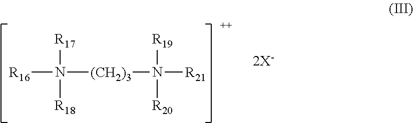

in which R₁₆ is an aliphatic radical containing from 16 to 30 carbon atoms, R₁₇, R₁₈, R₁₉, R₂₀ and R₂₁, which may be identical or different, are chosen from hydrogen and alkyl radicals containing from 1 to 4 carbon atoms, and X is an anion chosen from halides, acetates, phosphates, nitrates and methyl sulphates. Such diquaternary ammonium salts comprise, for example, propanetallowdiammonium dichloride;
quaternary ammonium salts containing at least one ester function, such as those of formula (IV) below:

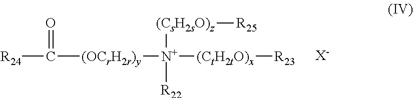

wherein:

R₂₂ is chosen from C₁-C₆ alkyl radicals and C₁-C₆ hydroxyalkyl or dihydroxyalkyl radicals;

R₂ is chosen from:
the radical

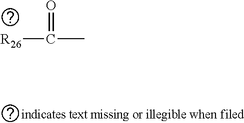

saturated or unsaturated, linear or branched C₁-C₂₂ hydrocarbon-based radicals R₂₇,
a hydrogen atom,

R₂₅ is chosen from:
the radical

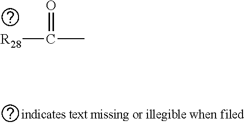

saturated or unsaturated, linear or branched C₁-C₆ hydrocarbon-based radicals R₂₉,
a hydrogen atom,

R₂₄, R₂₆ and R₂₈, which may be identical or different, are chosen from saturated or unsaturated, linear or branched C₇-C₂₁ hydrocarbon-based radicals;

r, s and t, which may be identical or different, are integers ranging from 2 to 6;

y is an integer ranging from 1 to 10;

x and z, which may be identical or different, are integers ranging from 0 to 10;

X— is a simple or complex, organic or inorganic anion;

with the proviso that the sum x+y+z is from 1 to 15, that, when x is 0, then R₂₃ denotes R₂₇, and that, when z is 0, then R₂₅ denotes R₂₉.

The alkyl radicals R₂₂ may be linear or branched, and according to at least one embodiment, is linear.

In at least one embodiment, R₂₂ is a methyl, ethyl, hydroxyethyl or dihydroxypropyl radical, and according to at least one further embodiment, is a methyl or ethyl radical.

According to at least one embodiment, the sum x+y+z is from 1 to 10.

When R₂₃ is a hydrocarbon-based radical R₂₇, it may be long and have from 12 to 22 carbon atoms, or short and have from 1 to 3 carbon atoms.

When R₂₅ is a hydrocarbon-based radical R₂₉, it has 1 to 3 carbon atoms.

According to at least one embodiment, R₂₄, R₂₆ and R₂₈, which may be identical or different, are saturated or unsaturated, linear or branched C₁₁-C₂₁ hydrocarbon-based radicals, and further, for example, from saturated or unsaturated, linear or branched C₁₁-C₂₁ alkyl and alkenyl radicals.

For instance, x and z, which may be identical or different, are 0 or 1.

According to at least one embodiment, y is equal to 1.

For example, r, s and t, which may be identical or different, are 2 or 3, and according to at least one embodiment are equal to 2.

The anion is, in at least one embodiment, chosen from a halide (chloride, bromide or iodide) and an alkyl sulphate, such as a methyl sulphate. However, it is possible to use the methanesulphonate, the phosphate, the nitrate, the tosylate, an anion derived from an organic acid, such as acetate or lactate, or any other anion compatible with ammonium comprising an ester function.

For example, as disclosed herein, the anion X— can be chloride or methyl sulphate.

In the composition according to the present disclosure, use is made, for instance, of the ammonium salts of formula (IV) wherein:
R₂₂ is a methyl or ethyl radical,
x and y are equal to 1;
z is equal to 0 or 1;
r, s and t are equal to 2;
R₂₃ is chosen from:
the radical

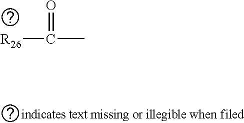

methyl, ethyl or C₁₄-C₂₂ hydrocarbon-based radicals,
a hydrogen atom;
R₂₅ is chosen from:
the radical

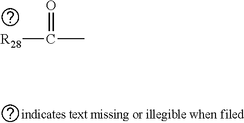

a hydrogen atom;
R₂₄, R₂₆ and R₂₈, which may be identical or different, are chosen from saturated or unsaturated, linear or branched C₁₃-C₁₇ hydrocarbon-based radicals, for example from saturated or unsaturated, linear or branched C₁₃-C₁₇ alkyl and alkenyl radicals.

According to at least one embodiment, the hydrocarbon-based radicals are linear.

Non-limiting mention may, for example, be made of the compounds of formula (IV) such as the salts (for instance, chloride or methyl sulphate) of diacyloxyethyldimethylammonium, of diacyloxyethylhydroxyethylmethylammonium, of monoacyloxyethyldihydroxyethylmethylammonium, of triacyloxyethylmethylammonium or of monoacyloxyethylhydroxyethyldimethylammonium, and mixtures thereof. The acyl radicals may contain, for example, from 14 to 18 carbon atoms and are derived, in at least one embodiment, from a plant oil such as palm oil or sunflower oil. When the compound contains several acyl radicals, these radicals may be identical or different.

These products are obtained, for example, by direct esterification of triethanolamine, triisopropanolamine, alkyldiethanolamine or alkyldiisopropanolamine, which are optionally oxyalkylenated, with fatty acids or with mixtures of fatty acids of plant or animal origin, or by transesterification of their methyl esters. This esterification is followed by a quaternization using an alkylating agent such as an alkyl (such as methyl or ethyl) halide, a dialkyl (such as methyl or ethyl) sulphate, methyl methanesulphonate, methyl para-toluenesulphonate, glycol chlorohydrin or glycerol.

Such compounds are, for example, sold under the names Dehyquart® by the company Henkel, Stepanquat® by the company Stepan, Noxamium® by the company Ceca or Rewoquat® WE 18 by the company Rewo-Witco.

The composition according to the present disclosure, may comprise a mixture of quaternary ammonium salts of mono-, di- and triester with a majority by weight of diester salts.

An example of a mixture of ammonium salts which may be used is the mixture containing 15% to 30% by weight of acyloxyethyldihydroxyethylmethylammonium methyl sulphate, 45% to 60% of diacyloxyethylhydroxyethylmethylammonium methyl sulphate and 15% to 30% of triacyloxyethylmethylammonium methyl sulphate, the acyl radicals containing from 14 to 18 carbon atoms and being derived from optionally partially hydrogenated palm oil.

It is also possible to use ammonium salts containing at least one ester function which are described, for instance, in U.S. Pat. Nos. 4,874,554 and 4,137,180.

Among quaternary ammonium salts of formula (I), consideration should be given to, on the one hand, tetraalkylammonium chlorides such as, for example, dialkyldimethylammonium or alkyltrimethylammonium chlorides, in which the alkyl radical contains from approximately 12 to 22 carbon atoms, for example, behenyltrimethylammonium, distearyldimethylammonium, cetyltrimethylammonium or benzyldimethylstearylammonium chloride, or, on the other hand, palmitylamidopropyttrimethylammonium chloride or stearamidopropyldimethyl(myristyl acetate)ammonium chloride sold under the name Ceraphyl® 70 by the company Van Dyk.

As disclosed herein, the cationic surfactants that may be used can be chosen from quaternary ammonium salts, and according to at least one embodiment, from cetyltrimethylammonium chloride, behenyltrimethylammonium chloride and palmitylamido-propyltrimethylammonium chloride.

The ionic surfactants that can be used in the cosmetic composition can also be anionic surfactants.

As anionic surfactants that can be used in the cosmetic composition according to the present disclosure, non-limiting mention may be made of salts, for example, alkali metal salts, such as sodium salts, ammonium salts, amine salts, amino alcohol salts or alkaline-earth metal salts, for example magnesium salts, of the following types: alkyl sulphates, alkyl ether sulphates, alkylamido ether sulphates, alkylarylpolyether sulphates, monoglyceride sulphates; alkyl sulphonates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphoacetates; acyl sarcosinates and acyl glutamates, the alkyl and acyl groups of all these compounds containing from 6 to 24 carbon atoms and, according to at least one embodiment, the aryl group is a phenyl or benzyl group.

Use may also be made of C₆-C₂₄ alkyl monoesters of polyglycoside dicarboxylic acids, such as alkyl glucoside citrates, alkyl polyglycoside tartrates and alkyl polyglycoside sulphosuccinates, alkyl sulphosuccinamates, acyl isethionates and N-acyl taurates, the alkyl or acyl group of all these compounds containing from 12 to 20 carbon atoms.

Another group of anionic surfactants that can be used in the compositions of the present disclosure are acyl lactylates, the acyl group of which contains from 8 to 20 carbon atoms.

In addition, non-limiting mention may be made of alkyl-D-galactosiduronic acids and their salts, and also polyoxyalkylenated (C₆-C₂₄)alkyl ether carboxylic acids, polyoxyalkylenated (C₆-C₂₄)alkyl (C₆-C₂₄)aryl ether carboxylic acids, polyoxyalkylenated (C₆-C₂₄)alkylamido ether carboxylic acids and their salts, such as those containing from 2 to 50 ethylene oxide units, and mixtures thereof.

Alkyl sulphates, alkyl ether sulphates and alkyl ether carboxylates, and mixtures thereof, can also be used, for example, in the form of alkali metal or alkaline-earth metal salts, ammonium salts, amine salts or amino alcohol salts.

According to at least one embodiment, the ionic surfactant is a cationic surfactant.

The nonionic surfactants that can be used in the cosmetic composition of the present disclosure are compounds that are well known per se (see, for example, in this regard “Handbook of Surfactants” by M. R. Porter, published by Blackie & Son (Glasgow and London), 1991, pp. 116-178). For instance, they are chosen from polyethoxylated, polypropoxylated or polyglycerolated fatty acids, (C₁-C₂₀)alkylphenols, alpha-diols or alcohols having a fatty chain containing, for example, from 8 to 18 carbon atoms, it being possible for the number of ethylene oxide or propylene oxide groups to range from 2 to 50 and for the number of glycerol groups to range from 2 to 30, for example.

Non-limiting mention may also be made of condensates of ethylene oxide and of propylene oxide with fatty alcohols; polyethoxylated fatty amides having, for example, from 2 to 30 units of ethylene oxide, polyglycerolated fatty amides containing on average from 1 to 5 glycerol groups, and for instance from 1.5 to 4 glycerol groups, ethoxylated fatty acid esters of sorbitan having from 2 to 30 units of ethylene oxide, fatty acid esters of sucrose, fatty acid esters of polyethylene glycol, (C₆-C₂₄ alkyl)polyglycosides, N—(C₆-C₂₄ alkyl)glucamine derivatives, amine oxides such as (C₁₀-C₁₄ alkyl)amine oxides or N—(C₁₀-C₁₄ acyl)aminopropylmorpholine oxides.

Among the nonionic surfactants mentioned above, polyethoxylated, polypropoxylated or polyglycerolated alcohols may be used, for example.

The surfactant(s) is (are) present in an amount ranging from 0.01% to 20% by weight, such as from 0.05% to 10% by weight, and even further, for example, at a concentration ranging from 0.1% to 5% by weight, relative to the total weight of the composition.

The cosmetic composition according to the present disclosure may further comprise at least one cosmetic adjuvant chosen from conditioners of ester type, antifoams, moisturizers, emollients, plasticizers, mineral thickening agents, organic thickening agents, which may or may not be polymeric and which may or may not be associative, silicone or nonsilicone, water-soluble and liposoluble sunscreens, permanent or temporary dyes, fragrances, peptizers, preserving agents, ceramides and pseudoceramides, vitamins and provitamins, including panthenol, proteins, sequestering agents, solubilizing agents, basifying agents, acidifying agents, anticorrosives, reducing agents or antioxidants, oxidizing agents, mineral fillers and flakes.

Those skilled in the art will take care to select the optional at least one cosmetic adjuvant and the amount thereof in such a way that they do not harm the properties of the compositions of the present disclosure.

According to at least one embodiment, the at least one cosmetic adjuvant is present in an amount ranging from 0.001% to 50% by weight, relative to the total weight of the composition.

The term “cosmetically acceptable medium” is understood to mean a medium compatible with keratin materials, such as the hair.

The cosmetically acceptable medium can be an alcoholic, aqueous or aqueous-alcoholic medium. Thus, the medium can comprise only water or a monoalcohol, or a mixture of water and at least one cosmetically acceptable monoalcohols such as C₁-C₄ lower alcohols, polyol ethers with a free hydroxyl, and mixtures thereof. According to at least one embodiment, the alcohol is ethanol.

According to another embodiment, the composition according to the present disclosure comprises water in an amount up to 99% by weight, for example, ranging from 60% to 99% by weight, relative to the total weight of the composition.

In the case where the composition as disclosed herein is a gel, the composition comprises water in an amount up to 99.8% by weight, for example in an amount ranging from 20% to 99% by weight, relative to the total weight of the composition.

The present disclosure also relates to a process for the cosmetic treatment of the hair, for example for hairstyling, which comprises applying an effective amount of a composition described above to dry or wet hair, and possibly in rinsing out after an optional leave-on time or after optional drying.

According to another embodiment, the composition is not a rinse-out composition.

The present disclosure also relates to the use of a cosmetic composition for fixing keratin fibers.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

The following examples are given by way of nonlimiting illustration of the present disclosure.

### EXAMPLES

The following compositions according to the present disclosure were prepared:

### Mousse

| | 1 | 2 |
|---|---|---|
| Lambda-carrageenan | 1.2% | 1.4% |
| (Satiagum UTC 10-Degussa) | | |
| Glycerol | 3% | 2% |
| Ionic or nonionic surfactant (Polysorbate 20) | 0.6% | — |
| Nonionic fixing polymer (PVP) | — | 3% |
| Denatured alcohol | — | 5% |
| Preserving agents, neutralizing agent, fragrance | qs | qs |
| Water | qs 100 | qs 100 |
| Propellant gas (hydrocarbons) | 4% | 4% |

This mousse resulted in the formation of a relatively non-friable film on hair.

### Hairstyling Gels

| | 1 | 2 | 3 | 4 |
|---|---|---|---|---|
| Lambda-carrageenan | 3.3% | 3.7% | 3.4% | 4.7% |
| (Welgeenan ED 1039-Eurogum) | | | | |
| Glycerol | 2% | — | — | — |
| Sorbitol | — | 5% | — | — |
| Cationic guar gum (Jaguar C13S) | — | 0.2% | — | — |
| Ionic or nonionic surfactant | — | 0.5% | — | — |
| (Polysorbate 20) | | | | |
| Ionic fixing polymer (PVP) | — | 3% | — | — |
| Denatured alcohol | 0.6% | 5% | — | 0.1% |
| Tapioca dextrin (Cristal Tex 626- | — | — | 1% | — |
| National Starch) | | | | |
| Glucose (N-Tack - National | — | — | 1% | — |
| Starch) | | | | |
| Crosslinked potato carboxymethyl | — | — | — | 2.4% |
| starch (Primojel - DMV) | | | | |
| Preserving agents, neutralizing | qs | qs | | |
| agent, fragrance | | | | |
| Water | qs 100 | qs 100 | qs 100 | qs 100 |

This hairstyling gel, when applied to hair, resulted in lasting fixing of the shape of the head of hair.

1. A cosmetic composition for fixing the hair, wherein the cosmetic composition comprises, in a cosmetically acceptable medium:
at least one lambda-carrageenan polysaccharide;
at least one ceramide;
at least one polyol containing in its structure at least 3 hydroxyl groups, other than carrageenans; and
at least one additive chosen from fatty substances and fixing polymers other than carrageenans;
and wherein the only carrageenan polysaccharide(s) in the composition is/are lambda-carageenan polysaccharide(s).

2. The cosmetic composition according to claim 1, wherein the at least one polyol is chosen from simple polyols, monosaccharides, disaccharides, oligosaccharides and polysaccharides other than carrageenans.

3. The cosmetic composition according to claim 2, wherein the at least one polyol is not a polymer.

4. The cosmetic composition according to claim 2, wherein the at least one polyol is chosen from glycerol and sorbitol.

5. The cosmetic composition according to claim 1, wherein the at least one polyol is present in the cosmetic composition in an amount ranging from 0.1% to 30% by weight, relative to the total weight of the composition.

6. The cosmetic composition according to claim 5, wherein the at least one polyol is present in the cosmetic composition in an amount ranging from 0.5% to 15% by weight, relative to the total weight of the composition.

7. The cosmetic composition according to claim 1, wherein the molecular weight (MW) of the at least one lambda-carrageenan polysaccharide ranges from 100,000 to 1,000,000.

8. The cosmetic composition according to claim 7, wherein the molecular weight (MW) of the at least one lambda-carrageenan polysaccharide ranges from 250,000 to 800,000.

9. The cosmetic composition according to claim 1, wherein the at least one lambda-carrageenan polysaccharide is present in the cosmetic composition in an amount ranging from 0.1% to 30% by weight, relative to the total weight of the composition.

10. The cosmetic composition according to claim 9, wherein the at least one lambda-carrageenan polysaccharide is present in the cosmetic composition in an amount ranging from 0.5% to 15% by weight, relative to the total weight of the composition.

11. The cosmetic composition according to claim 1, further comprising at least one cosmetic adjuvant chosen from conditioners of ester type, antifoams, moisturizers, emollients, plasticizers, mineral thickening agents, organic thickening agents, which may or may not be polymeric and which may or may not be associative, silicone or nonsilicone, water-soluble and liposoluble sunscreens, permanent or temporary dyes, fragrances, peptizers, preserving agents, vitamins and provitamins, proteins, sequestering agents, solubilizing agents, basifying agents, acidifying agents, anticorrosives, reducing agents or antioxidants, oxidizing agents, mineral fillers and flakes.

12. The cosmetic composition according to claim 1, wherein the cosmetically acceptable medium is an aqueous, alcoholic or aqueous-alcoholic medium.

13. The cosmetic composition according to claim 1, wherein the cosmetic composition comprises water in an amount up to 99% by weight, relative to the total weight of the composition.

14. The cosmetic composition according to claim 13, wherein the cosmetic composition comprises water in an amount ranging from 60% to 99% by weight, relative to the total weight of the composition.

15. A cosmetic treatment process, comprising:
applying a cosmetic composition to keratin fibers;
wherein the cosmetic composition comprises, in a cosmetically acceptable medium:
at least one lambda-carrageenan polysaccharide;
at least one ceramide;
at least one polyol containing in its structure at least 3 hydroxyl groups, other than carrageenans; and
at least one additive chosen from silicones, fatty substances, and fixing polymers other than carrageenans;
and wherein the only carrageenan polysaccharide(s) in the composition is/are lambda-carageenan polysaccharide(s).

16. The cosmetic treatment process according to claim 15, wherein after the cosmetic composition is applied, there is no rinsing step.

17. A method for fixing keratin fibers comprising:
applying a cosmetic composition to keratin fibers,
wherein the cosmetic composition comprises, in a cosmetically acceptable medium:
at least one lambda-carrageenan polysaccharide;
at least one ceramide;
at least one polyol containing in its structure at least 3 hydroxyl groups, other than carrageenans; and
at least one additive chosen from silicones, fatty substances, and fixing polymers other than carrageenans;
and wherein the only carrageenan polysaccharide(s) in the composition is/are lambda-carageenan polysaccharide(s).

18. A method for caring for keratin fibers comprising:
applying a cosmetic composition to keratin fibers such as the hair;
wherein the cosmetic composition comprises, in a cosmetically acceptable medium:
at least one lambda-carrageenan polysaccharide;
at least one ceramide;
at least one polyol containing in its structure at least 3 hydroxyl groups, other than carrageenans; and
at least one additive chosen from silicones, fatty substances, and fixing polymers other than carrageenans;
and wherein the only carrageenan polysaccharide(s) in the composition is/are lambda-carageenan polysaccharide(s).

19. The cosmetic composition according to claim 1, wherein the total amount of polyol containing in its structure at least 3 hydroxyl groups, other than carrageenans, in the composition ranges from 2 to 5%.
